(19) United States
(12) Patent Application Publication
(10) Pub. No.: US 2023/0346227 A1
(43) Pub. Date: Nov. 2, 2023

(54) SYSTEMS AND METHODS FOR PERFORMING DIGITAL SUBTRACTION ANGIOGRAPHY USING THERMAL IMAGING

(71) Applicant: Vikas Khurana, BROOMALL, PA (US)

(72) Inventor: Vikas Khurana, BROOMALL, PA (US)

(21) Appl. No.: 17/928,610

(22) PCT Filed: Jun. 1, 2021
(86) PCT No.: PCT/US2021/035080

§ 371 Date: Nov. 29, 2022

Related U.S. Application Data
(60) Provisional application No. 63/033,152, filed on Jun. 1, 2020.

(51) Int. Cl. A61B 5/01; A61B 5/026; A61K 41/00

(52) U.S. Cl. CPC A61B 5/015; A61B 5/0261; A61K 41/0052

(57) ABSTRACT

The present embodiment discloses a method of performing digital subtraction thermography by employing the technique of digital subtraction angiography (DSA). The method includes acquiring a pre-contrast thermal image of a target area; application of thermally controlled intravascular fluid as a contrast agent onto the target area; acquiring a post-contrast thermal image; acquiring a plurality of post-contrast thermal images after a fixed time interval; processing of the pre-contrast thermal image, the post-contrast thermal image and the plurality of post-contrast thermal images; and generating a thermogram.

### FIELD OF THE INVENTION

The present embodiment generally relates to a method for performing digital subtraction angiography and more particularly relates to the method for performing digital subtraction angiography of the anatomy of a subject by employing thermal imaging using several methods and systems for producing a temperature differential in the anatomy.

### BACKGROUND OF THE INVENTION

Medical imaging is a technique of creating anatomical maps of the surface or interior of a body for the diagnosis of clinical manifestations. Various imaging techniques include X-ray, CT scan, radiography, Magnetic resonance imaging (MRI), endoscopy, ultrasound, thermal imaging and nuclear medicine scans.

Digital Subtraction Angiography (DSA) produces images of a subject's blood vessels as the difference image between a post- and a pre-contrast injection images. Since the contrast medium injected flows only in the vessels, the image data arising from other structures does not change in the two images and is eliminated by the subtraction.

Thermal imaging is a technique for creating an image based on detecting the infrared rays emitted. Thermal imaging is based on the principle of detecting temperature variations related to the blood flow. Traditionally, thermal imaging was based on the principle that every object emits heat (infrared rays), which are sensed by the thermal imaging camera and a thermal image is generated related to differential emission. For example, cancer cells have a tendency to multiply rapidly and therefore require more blood supply. Thus, the area having an abnormal growth will have a higher temperature compared to the normal body temperature and thus will generate higher infrared emission. This technique has disadvantages vis-a-vis false positive test and low sensitivity.

A contrast agent is used in DSA to enhance the contrast between the regions having higher blood flow and the regions having normal blood flow. The contrast agent heightens the contrast, thereby allowing for better visualization of the abnormalities. The contrast agents being used currently in clinical medicine are biopharmaceuticals and have significant toxicity for subjects. Traditional contrast agents like iodine or gadolinium-containing contrast agents cannot be used repeatedly and can cause significant side effects in subjects with allergic reactions, subjects with diabetes or renal failure. There exists no imaging methodology with minimal or no side effects for a contrast agent, which is not limited by the dose and can be repeatedly used. Another drawback of using current contrast agents is the exposure to radiation and gamma rays which are used to visualize the passage of contrast material through the body. In addition, there exists no technique for employing thermal imaging in digital subtraction angiography. Furthermore, the contrast agent has limitations pertaining to the inability of being applied to open wounds.

Thus, there exists a need for developing a technique for employing thermal imaging in digital subtraction angiography (DSA) for enhancing the contrast between the area of higher blood flow and the area of lower blood flow in different anatomical areas of the body.

### SUMMARY OF THE INVENTION

As mentioned in the foregoing, the embodiment herein provides a method for employing thermal imaging in digital subtraction angiography. The embodiment provides cooling or warming of the thermal contrast agent for enhancing the contrast differential between the regions of higher blood flow and the regions of lower blood flow. The current contrast agents used in radiology and body imaging are only mechanical in nature and by design are not supposed to cause any pathophysiological change in the body. The change in temperature stimulates pathophysiological correction in the body and thus with a thermal contrast agent we can evaluate the ability of the tissue to provide pathophysiological correction to the temperature change.

In an aspect, a method of performing digital subtraction angiography for enhancing contrast of an anatomical area of the body is provided. The method includes acquiring a pre-contrast thermal image or the mask image of a target area. In an embodiment, a video and a time-lapse photograph are recorded in real-time; injecting a temperature adjusted IV fluid into a blood vessel at a pre determined speed; acquiring a post-contrast thermal image immediately after the injection of the IV fluid; acquiring a plurality of post-contrast thermal images after a fixed time interval; processing of the pre-contrast thermal image, the post-contrast thermal image and the plurality of post contrast thermal images; generating a thermogram for determining the relationship between the temperature variations with the blood flow.

The method further includes comparing and superimposing the post-contrast thermal image and the plurality of post-contrast thermal images with the pre-contrast thermal image; subtracting the superimposed portions of the post-contrast thermal image and the plurality of post-contrast thermal images with the pre-contrast thermal image; determining a change in the rate of temperature recovery of the target area; analyzing the thermogram for assessing the pathophysiological state of the individual.

In another aspect, a method of performing digital subtraction angiography for enhancing contrast differential or thermal contrast differential of an inflamed region on the surface of intact skin is provided. The method includes acquiring a pre-contrast thermal image or a mask image of a target area. In an embodiment, a video and a time-lapse photograph are recorded in real-time; spraying of an evaporative fluid onto the target area; acquiring a post-contrast thermal image after the evaporation of the evaporative fluid; acquiring a plurality of post-contrast thermal images after a fixed time interval; processing of the pre-contrast thermal image, the post-contrast thermal image and the plurality of thermal images; generating a thermogram for determining the relationship between the temperature variations with the blood flow.

The method further includes comparing and superimposing the post-contrast thermal image and the plurality of post-contrast thermal images with the pre-contrast thermal image; subtracting the superimposed portions of the post-contrast thermal image and the plurality of post-contrast thermal images with the pre-contrast thermal image; determining a change in the rate of temperature recovery of the target area; analyzing the thermogram for assessing the pathophysiological state of the individual.

In yet another aspect, a method of performing digital subtraction angiography for enhancing a contrast of a moist and a wet infected area of the body is provided. The method includes acquiring a first pre-contrast thermal image ora mask image of the target area. In an embodiment, a video and a time-lapse photograph are recorded in real-time; placing a transparent barrier over the target area; acquiring a second pre-contrast thermal image after the temperature recovery of the target area; spraying of an evaporative fluid over the barrier; acquiring a post-contrast thermal image after the evaporation of the evaporative fluid; processing of the first pre-contrast thermal image, the second pre-contrast thermal image and the post-contrast thermal image; obtaining the thermographic data corresponding to a plurality of points of the target area; generating a composite thermogram for determining the relationship between the temperature variations with the blood flow.

The method further includes comparing and superimposing the first pre-contrast thermal image, the second pre-contrast thermal image and the post-contrast thermal image; subtracting the superimposed portions of the first pre-contrast thermal image, the second pre-contrast thermal image and the post-contrast thermal image; analyzing the composite thermogram for assessing the pathophysiological state of the individual.

In another aspect, a method for determining the pathophysiological state of an individual is provided. The method includes: measuring of the heart rate using a device having a camera; acquiring a thermal image; and determining the pathophysiological state by analyzing the heart rate and the thermal images.

In another aspect, a system for determining the pathophysiological state of an individual is provided. The system includes a pulse reading module, a thermal imaging module, a quantification module and an output module. The pulse reading module is capable of determining the heart rate. The thermal imaging module is capable of capturing the thermal images of the target area. The quantification module communicates with the pulse reading module and the thermal imaging module and is capable of processing the heart rate and the thermal images for determining the pathophysiological state of the person. The output module communicates with the quantification module and is capable of displaying the pathophysiological state of the person.

The preceding is a simplified summary to provide an understanding of some aspects of embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

The above and still further features and advantages of embodiments of the present invention will become apparent upon consideration of the following detailed description of embodiments thereof, especially when taken in conjunction with the accompanying drawings, and wherein:

FIG. 1 illustrates a method (100) of performing digital subtraction angiography for the anatomical areas of the body, according to an embodiment herein;

FIG. 2 illustrates a method (200) of performing digital subtraction angiography for the inflamed region on the surface of the skin, according to an embodiment herein;

FIG. 3 illustrates a method (300) of performing digital subtraction angiography for the moist and wet infected area of the body, according to an embodiment herein;

FIG. 4 illustrates a method (400) for determining the pathophysiological state of an individual, according to an embodiment herein;

FIG. 5 illustrates a system (500) for determining the pathophysiological state of an individual, according to an embodiment herein;

FIG. 6 illustrates morphology of two PPG waves, according to an embodiment herein; and

FIG. 7 illustrates traces of a PPG wave from hypothetical subject, according to an embodiment herein.

To facilitate understanding, like reference numerals have been used, where possible, to designate like elements common to the figures.

### DETAILED DESCRIPTION OF THE PREFERRED EMBODIMENTS

As used throughout this application, the word “may” is used in a permissive sense (i.e., meaning having the potential to), rather than the mandatory sense (i.e., meaning must). Similarly, the words “include”, “including”, and “includes” mean including but not limited to.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term IV refers to Intravascular fluid.

The term “subject” refers to a subject, or an organ or tissue that is targeted.

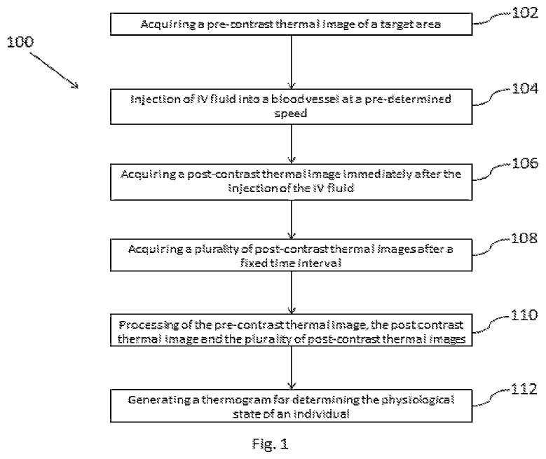

FIG. 1 illustrates the method (100) of performing digital subtraction angiography, according to an embodiment. Digital subtraction angiography is an imaging technique for visualizing the blood vessels of the bones, muscles and other tissues of the body. In an embodiment, the digital subtraction angiography is employed during surgery. In an embodiment, the digital subtraction angiography visualizes the blood vessels of the internal organs of the body during the surgery. The method (100) of performing digital subtraction angiography includes following steps as described herein.

At step 102, a pre-contrast thermal image is acquired of a target area. In an embodiment, the pre-contrast thermal image is a mask image. In an embodiment, the pre-contrast thermal image is an image captured before the injection of a thermal contrast agent. In an embodiment, the precontrast thermal image serves as a standard for assessing the pathophysiological state of a subject. In an embodiment, the target area is an anatomical area of the body. In a preferred embodiment, the target area is a bone, muscle and other tissues of the body. In an embodiment, the digital subtraction angiography is employed during surgery. In an embodiment, the digital subtraction angiography is done for the internal organ of the body.

In an embodiment, the pre-contrast thermal image is acquired by an infrared camera or another device for recording infrared rays. In an embodiment, the body emits heat in the form of infrared rays. In an embodiment, the infrared rays are detected by the infrared camera and the pre-contrast thermal image is captured.

In an embodiment, a video and a time-lapse photograph are recorded in real-time. In an embodiment, the pre-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to a surrounding tissue.

At step 104, a temperature specified IV fluid is injected into a blood vessel at a pre-determ fined speed. In an embodiment, the IV fluid includes fluid such as, but not limited to, ringer lactate, saline and dextrose solution. In a preferred embodiment, the IV fluid is a saline. In an embodiment, the IV fluid is the thermal contrast agent.

The IV fluid is injected into the blood vessel. In an embodiment, the IV fluid is injected into a vein. In another embodiment, the IV fluid is injected into an artery. In an embodiment, the IV fluid is injected intravenously, in antecubital or large forearm vein, hand veins, foot veins, chest ports, central lines and cannulas inserted into internal and external jugular vein.

In an embodiment, the IV fluid is injected through a pressure injection, a catheter, 18-gauge needle or 20-gauge needles. In an embodiment, the pressure injection is capable of selecting the amount of IV fluid injected, pressure, flow and rate of the IV fluid inside the subject. In an embodiment, the pressure injection is capable of preventing complications such as, but not limited to, contrast extravasation, sepsis and air embolism. In an embodiment, the IV fluid is injected through a catheter attached onto an instrument used in endoscopy, laparoscopy, surgery, intravascular procedure, intracranial procedure, intracardiac procedure, peritoneal imaging, peritoneal scope, bronchoscopy and pleural biopsies.

In an embodiment, the IV fluid is injected inside the artery during endoscopy, laparoscopy, surgery, intracranial procedure, intracardiac procedure, peritoneal imaging, peritoneal scope, bronchoscopy and pleural biopsies. In another embodiment, the cold IV fluid is injected inside the vein during endoscopy, laparoscopy, surgery, intracranial procedure, intracardiac procedure, peritoneal imaging, peritoneal scope, bronchoscopy and pleural biopsies.

In an embodiment, the volume of the IV fluid injected inside the subject is 500 ml. In an embodiment, the volume of the cold IV fluid injected inside the subject depends on the height, weight, sex, age, cardiac output of the subject. In an embodiment, the total volume of the cold IV fluid is injected inside the subject without a break. In another embodiment, the total volume of the IV fluid is injected inside the subject in multiple phases.

In an embodiment, the IV fluid is injected inside the subject at a speed ranging from 60 to 260 ml/min. In an embodiment, the speed of the IV fluid depends on the site of injection and the target area. In an embodiment, the speed of the IV fluid during cardiac catheterization is 2 ml/min.

In an embodiment, the IV fluid has a temperature higher than the normal body temperature. In another embodiment, the IV fluid has a temperature lower than the normal body temperature.

In an embodiment, a thermal contrast agent is injected inside a vein. In an embodiment, the thermal contrast agent is injected directly into the artery by the surgeon. In an embodiment, the thermal contrast agent accumulates in a perivascular space, a bone fluid space and a bone. In an embodiment, the thermal contrast agent accumulates in the perivascular space within a time span of a few seconds. In an embodiment, the thermal contrast agent accumulates in the bone fluid space within a time span of few minutes. In an embodiment, the thermal contrast agent diffuses out from the accumulation space in the bone within few minutes. In an embodiment, the accumulation of the thermal contrast agent in the perivascular space allows assessment of the region of interest. In an embodiment, the accumulation of the thermal contrast agent in the bone fluid space allows contrasting images of the bone and the surrounding tissue. In an embodiment, the accumulation of the thermal contrast agent in the bone fluid space enables determining the intravascular and the extravascular activity. In an embodiment, the accumulation of the thermal contrast agent in the bone enables the detection of the affected bony structures. In an embodiment, the injection of the thermal contrast agent enables the diagnosis of the diseases of the bone. In a preferred embodiment, the thermal contrast agent helps in the diagnosis of osteomyelitis. In another embodiment, a negative thermal contrast is observed indicating an obstruction or blood clot within blood vessel lumen thus allowing identification or diagnosis of blocked blood vessel, deep venous thrombosis (DVT) or hollow lumen such as GI tract, spinal fluid flow, air flow in lungs etc.

In an embodiment, the IV fluid is chilled to a temperature ranging from 4-30° C. In an embodiment, the IV fluid injected inside the vein is colder than the IV fluid injected inside the artery. In an embodiment, the IV fluid injected inside the vein is transferred to heart. The heart is capable of thermo-diluting the cold IV fluid, thereby creating a requirement of decreased temperature or thermal contrast in the circulation. A dual contrast is obtained by injecting cold IV fluid inside vein and a hot IV fluid inside artery. In a preferred embodiment, the IV fluid having a temperature of 4° C. is injected inside the vein and the IV fluid having a temperature of 40° C. is injected inside the artery. For example, during skin grafting, the cold IV fluid is injected in the vein and fluid at a temperature of 40° C. is injected inside the artery supplying the skin to give dual contrast to enhance required features.

In an embodiment, the IV fluid is heated to a temperature ranging up to 45° C. In an embodiment, the temperature recovery of the IV fluid is observed. In an embodiment, the hot IV fluid causes pathophysiological changes of the blood vessels.

In an embodiment, the injection of the cold IV fluid and the hot IV fluid at the same site separated by time creates a thermal contrast. In an embodiment, the thermal contrast allows the visualisation of the blood flow of the bones, muscles and other tissues of the body. In an embodiment, the thermal contrast allows the visualisation of the blood flow of the internal organs of the body during the surgery. In an embodiment, the thermal contrast is established between the target area and the surrounding tissues. In an embodiment, the thermal difference induced pathophysiological changes in the target area. In an embodiment, the thermal difference induced pathophysiological changes between the target area and the surrounding tissues.

At step 106, a post-contrast thermal image is acquired immediately after the injection of the IV fluid. In an embodiment, the post-contrast thermal image is an image captured after the injection of the thermal contrast agent. In an embodiment, the thermal contrast is observed with respect to the pre-contrast thermal image and the post-contrast thermal image.

In an embodiment, the post-contrast thermal image is acquired by the infrared camera. In an embodiment, the body emits heat in the form of infrared rays. In an embodiment, the infrared rays are detected by the infrared camera and the post-contrast thermal image is captured.

In an embodiment, the video and the time-lapse photograph are recorded in real-time. In an embodiment, the post-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to the surrounding tissue.

At step 108, a plurality of post-contrast thermal images is acquired. In an embodiment, the plurality of post-contrast thermal images is acquired after a fixed time interval. In an embodiment, the plurality of post-contrast thermal images is acquired at a time interval ranging from 2 seconds to 5 minutes. In an embodiment, the plurality of post-contrast thermal images is acquired successively as the temperature of the target area normalizes. In an embodiment, the temperature recovery of the target region is observed through the plurality of post-contrast thermal images. In an embodiment, the temperature recovery is used for assessing the pathophysiological state of a subject.

In an embodiment, the plurality of post-contrast thermal images is acquired by the infrared camera. In an embodiment, the body emits heat in the form of infrared rays. In an embodiment, the infrared rays are detected by the infrared camera and the plurality of post-contrast thermal images is captured.

In an embodiment, the video and the time-lapse photograph are recorded in real-time. In an embodiment, the plurality of post-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows better visualization of the target area in relation to the surrounding tissue.

At step 110, the pre-contrast thermal image, the post-contrast thermal image and the plurality of post-contrast thermal images are processed. In an embodiment, the target area emits infrared rays. In an embodiment, the target area having a higher blood supply will emit more infrared rays than the area having a lower blood supply. For example, the target area having a tumour will require more supply of blood for its rapid growth as compared to the target area having normal blood supply. The target area having a tumour will emit more infrared rays as compared to the target area having normal blood supply. The target area having a tumour will normalize the temperature of the IV fluid more rapidly than the target area having normal blood supply.

In an embodiment, the pre-contrast thermal image, the post-contrast thermal image and the plurality of post-contrast thermal image are compared and superimposed. In an embodiment, the superimposed regions of the pre-contrast thermal image, the post-contrast thermal image and the plurality of post-contrast thermal image are subtracted from each other to generate a thermogram.

At step 112, the thermogram is generated. The thermogram is the thermal image generated by the infrared camera. The infrared camera detects the infrared rays, converts the infrared rays into an electrical signal and displays the result as thermal image. In an embodiment, the thermogram is generated by superimposing the pre-contrast thermal image, the post-contrast thermal image and the plurality of post-contrast thermal image. In an embodiment, the superimposed regions are subtracted from the pre-contrast thermal image, the post-contrast thermal image and the plurality of post-contrast thermal image. In an embodiment, the rate of temperature recovery of the target area is observed. In an embodiment, the thermal emissivity characteristic of the tissue is used for assessing the pathophysiological state of the specific tissue. In an embodiment, the heat emitted from the target area is used for assessing the pathophysiological state of the individual. In an embodiment, the thermogram is analyzed for assessing the pathophysiological state of the individual. In an embodiment, the pathophysiological state of the individual includes conditions such as, but not limited to, a tumor, an inflammation, an ischemia, and any growth of cells inside the body. In an embodiment, the heat emitted from the target area is used for assessing the pathophysiological state of the tissue. In an embodiment, the pathophysiological state of the tissue includes conditions such as, but not limited to, a tumor, an inflammation, an ischemia, a cyst and any growth of cells inside the body. For example, the spraying of the cold IV fluid on the cyst allows differentiating the cyst from a tumor. In an embodiment, the tumor has higher blood flow. In an embodiment, the cyst has limited or no blood flow. The tumor will emit more infrared rays as compared to the cyst having no blood supply. The tumor will normalize the temperature of the cold IV fluid more rapidly than the cyst. The tumor will normalize the temperature of the evaporating fluid more rapidly than the cyst.

In an embodiment, the thermal emissivity characteristic of the body is used for assessing the pathophysiological state of the subject. In an embodiment, the individual has a core temperature different than the normal body temperature. In an embodiment, during fever, the core temperature of the individual is higher than the normal body temperature. In an embodiment, the body of the individual absorb, transmit and reflect infrared radiations. In an embodiment, the contrast is established between the infrared rays emitted from the target area and the infrared rays absorbed, transmitted and reflected by the surrounding tissues. In an embodiment, the individual with deranged core body temperature exposed to cold air will have differential pathophysiological recovery. In an embodiment, the individual with deranged core body temperature who has taken antipyretic medications when exposed to cold air will have differential pathophysiological recovery. In an embodiment, the individual with deranged core body temperature exposed to cold air will have differential pathophysiological recovery related to other pathophysiological parameters like pulse rate and pulse morphology. In an embodiment, pulse morphology is determined to diagnose pathophysiological state of a subject. In an embodiment, the thermogram is generated through a multi spectral dynamic imaging (MSX) technique. The multi spectral dynamic imaging (MSX) technique is based on FLIR processors for acquiring thermal images and videos in real time. In an embodiment, the multi spectral dynamic imaging (MSX) technique adds visible light to the thermal images. In an embodiment, the visible light allows the better visualisation of the target area in relation to the surrounding tissue.

In an embodiment, the method (100) is employed for calculating the circulatory time. In an embodiment, the method (100) is employed for determining the velocity of the blood flow. In an embodiment, the method (100) is used for assessing the pathophysiological state of the subject based on the velocity of the blood flow. In an embodiment, the pathophysiological state of the subject includes conditions such as, but not limited to, fever, anemia, cardiac problems, polycythemia or myxedema, and conditions of other internal organs.

For example, the method (100) allows differentiating between normal heart function and congestive heart failure. The IV fluid is injected inside the vein, and the IV fluid is returned in the artery on the contralateral side. The flow of the IV fluid helps in determining the recovery of the heart during surgery. In an embodiment, during congenital heart defects, the blood from the vein goes to the right side and gets shunted to the left side through the hole in the septum of the heart and comes back into the circulation lot faster than when it goes from the heart to the lungs, back to the heart and then to the circulatory system.

In an embodiment, the heart rate of the individual is measured for determining the core body temperature of an individual. The heart rate lies in the range from 60-100 beats per minute. In an embodiment, the heart rate increases when the temperature of the individual is increased than the normal body temperature. In an embodiment, 1 degree increase in the body temperature leads to an increase in the heart rate by 10 beats per minute.

In an embodiment, the heart rate is measured by a device having a camera. In an embodiment, the device includes a mobile phone, a smart watch and a photographic camera. In an embodiment, the heart rate is measured by a non-contact optical technique of photoplethysmography (PPG). In an embodiment, the photoplethysmography (PPG) is used to detect volumetric changes in the blood flow. In an embodiment, the photoplethysmography (PPG) is based on the principle that the blood absorbs more light than the surrounding tissues. In an embodiment, the blood flow affects the reflection of light. In an embodiment, the blood flow is different in systole and diastole. In an embodiment, the blood flow as a function of time is different in different pathophysiological states.

In an embodiment, the heart rate is measured by placing a finger on the camera of the device. In an embodiment, the flash light of the devices serves as the light source in the visible range for reflection by the blood cells of the individual. In an embodiment, the light reflected is different in systole and diastole. In an embodiment, the blood flow as a function of time is different in different pathophysiological states.

In an embodiment, the method (100) is used for determining the total circulation time for subjects such as liver and kidney during surgery. The circulation time is determined by injecting the IV fluid in the arterial side and determining the time for the course aligned to exist in the venous site. In an embodiment, the method (100) is used in liver and kidney organ transplants for detecting vascular damage during transplantation. In an embodiment, determining the vascular damage prevents long-term rejection and failure of the transplanted organ.

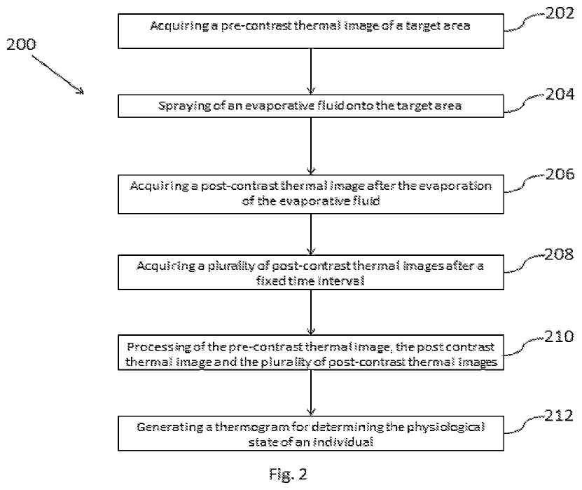

FIG. 2 illustrates the method (200) of performing digital subtraction angiography, according to an embodiment. The digital subtraction angiography is an imaging technique for visualizing the blood vessels of an intact, superficial tissue of the skin of the individual. The method (200) of performing digital subtraction angiography includes:

At step 202, a pre-contrast image is acquired of a target area. In an embodiment, the precontrast thermal image is a mask image. In an embodiment, the pre-contrast thermal image is an image captured before the spraying of a thermal contrast agent. In an embodiment, the precontrast thermal image serves as a standard for assessing the pathophysiological state of a subject. In an embodiment, the target area is an intact, superficial tissue of the skin of the individual. In a preferred embodiment, the target area is the inflamed region of the skin.

In an embodiment, the pre-contrast thermal image is acquired by an infrared camera. In an embodiment, the body emits heat in the form of infrared rays. In an embodiment, the infrared rays are detected by the infrared camera and the pre-contrast thermal image is captured.

In an embodiment, a video and a time-lapse photograph are recorded. In an embodiment, the pre-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to a surrounding tissue.

At step 204, an evaporative fluid is sprayed onto the target area. In a preferred embodiment, the evaporative fluid is the thermal contrast agent. In an embodiment, the evaporative fluid is a mixture of alcohol and water. In a preferred embodiment, the evaporative fluid contains 65% alcohol and 35% water. In an embodiment, the mixture of alcohol and water is sprayed on the intact, superficial tissue of the skin of the individual. In another embodiment, the evaporative fluid is saline. In an embodiment, the cold saline is sprayed onto the target area during a surgery. In an alternative embodiment, the subject may be exposed to hot air to raise the temperature of the subject such as by using a hair dryer.

In an embodiment, the volume of the evaporative fluid sprayed onto the target area lies in the range of 15 to 30 ml. In an embodiment, the volume of the evaporative fluid is sufficient enough to cover the plantar and dorsal side of the target area. In an embodiment, the volume of the fluid is sufficient to submerge the subject and thermal image of the submerged subject is obtained.

In an embodiment, the evaporative fluid is used to establish the contrast between the areas of higher blood supply and the areas of lower blood supply. In an embodiment, the evaporative fluid is used to establish the contrast between the target area and the surrounding tissues.

At step 206, a post-contrast thermal image is acquired after the evaporation of the evaporative fluid. In an embodiment, the post contrast thermal image is an image captured after the spraying of the evaporative fluid. In an embodiment, the thermal contrast is observed with respect to the pre-contrast thermal image and the post-contrast thermal image.

In an embodiment, the evaporation of the evaporative fluid depends on the factors such as, but not limited to, temperature of the room, humidity of the room and evaporation of water. In a preferred embodiment, the post-contrast thermal image is acquired after a pre-determ fined time. In an embodiment, the post-contrast thermal image is acquired after the complete evaporation of the evaporative fluid.

In an embodiment, the post-contrast thermal image is acquired by the infrared camera. In an embodiment, the body emits heat in the form of infrared rays. In an embodiment, the infrared rays are detected by the infrared camera and the post-contrast thermal image is captured.

In an embodiment, the video and the time-lapse photograph are recorded. In an embodiment, the post-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to the surrounding tissue.

At step 208, a plurality of post-contrast thermal images is acquired. In an embodiment, the plurality of post-contrast thermal images is acquired after a fixed time interval. In an embodiment, the plurality of post-contrast thermal images is acquired at a time interval ranging from 2 seconds to 5 minutes. In an embodiment, the plurality of post-contrast thermal images is acquired successively as the temperature of the target area normalizes. In an embodiment, the temperature recovery of the target region is observed through the plurality of post-contrast thermal images.

In an embodiment, the plurality of post-contrast thermal images is acquired by the infrared camera. In an embodiment, the body emits heat in the form of infrared rays. In an embodiment, the infrared rays are detected by the infrared camera and the plurality of post-contrast thermal images is captured.

In an embodiment, the video and the time-lapse photograph are recorded. In an embodiment, the plurality of post-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to the surrounding tissue.

At step 210, the pre-contrast thermal image, the post-contrast thermal image and the plurality of post-contrast thermal images are processed. In an embodiment, the target area emits infrared rays. In an embodiment, the target area having a higher blood supply will emit more infrared rays than the area having a lower blood supply. For example, the target area having an inflammation will require more supply of blood as compared to the target area having normal blood supply. The target area having an inflammation will emit more infrared rays as compared to the target area having normal blood supply. The target area having an inflammation will normalize the temperature of the evaporative fluid more rapidly than the target area having normal blood supply.

At step 212, a thermogram is generated. The thermogram is the thermal image generated by the infrared camera. The infrared camera detects the infrared rays, converts the infrared rays into an electrical signal and displays the result as a thermal image. In an embodiment, the thermogram is generated by superimposing the pre-contrast thermal image, the post-contrast thermal image and the plurality of the post-contrast thermal image. In an embodiment, the superimposed regions are subtracted from the pre-contrast thermal image, the post-contrast thermal image and the plurality of post-contrast thermal image. In an embodiment, the rate of temperature recovery of the target area is observed. In an embodiment, the thermogram is analyzed for assessing the pathophysiological state of the individual. In an embodiment, the pathophysiological state of the individual includes conditions such as, but not limited to inflammation, abscess or a boil on the intact skin. In another embodiment, the pathophysiological state of the individual includes conditions such as fever, dehydration and sepsis. For example, during fever, the temperature recovery of the body will be faster. In an embodiment, during fever, the core temperature of the body is higher than the normal body temperature. The individual will emit more infrared rays during fever than in the normal state. The evaporative fluid will evaporate faster during fever than in the normal state.

In an embodiment, the thermal emissivity characteristic of the body is used for assessing the pathophysiological state of the individual. In an embodiment, the individual has a core temperature different than the normal body temperature. In an embodiment, during fever, the core temperature of the individual is higher than the normal body temperature. In an embodiment, the body of the individual absorb, transmit and reflect infrared radiations. In an embodiment, the contrast is established between the infrared rays emitted from the target area and the infrared rays absorbed, transmitted and reflected by the surrounding tissues.

In an embodiment, the thermogram is generated through a multi spectral dynamic imaging (MSX) technique. The multi spectral dynamic imaging (MSX) technique is based on FLIR processors for acquiring thermal images and videos in real time. In an embodiment, the multi spectral dynamic imaging (MSX) technique adds visible light to the thermal images. In an embodiment, the visible light allows the better visualization of the target area in relation to the surrounding tissue.

In an embodiment, the heart rate of the individual is measured for determining the core body temperature of an individual. The heart rate lies in the range from 60-100 beats per minute. In an embodiment, the heart rate increases when the temperature of the individual is increased than the normal body temperature. In an embodiment, 1 degree increase in the body temperature leads to an increase in the heart rate by 10 beats per minute.

In an embodiment, the heart rate is measured by a device having a camera. In an embodiment, the device includes a mobile phone, a smart watch and a photographic camera. In an embodiment, the heart rate is measured by an optical technique of photoplethysmography (PPG). In an embodiment, the photoplethysmography (PPG) is used to detect volumetric changes in the blood flow. In an embodiment, the photoplethysmography (PPG) is based on the principle that the blood absorbs more light than the surrounding tissues. In an embodiment, the blood flow affects the reflection of light. In an embodiment, the blood flow is different in systole and diastole.

In an embodiment, the heart rate is measured by placing a finger on the camera of the device. In an embodiment, the flash light of the devices serves as the light source in the visible range for reflection by the blood cells of the individual. In an embodiment, the light reflected is different in systole and diastole.

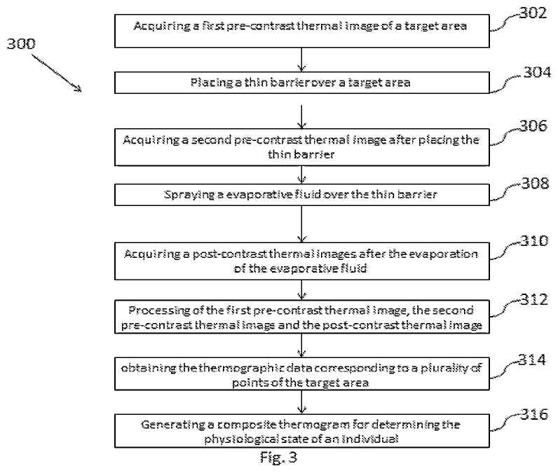

FIG. 3 illustrates the method (300) of performing digital subtraction angiography, according to an embodiment. The digital subtraction angiography is an imaging technique for visualising the blood vessels of a wet and a moist infected area of the body. The method (300) of performing digital subtraction angiography includes:

At step 302, a first pre-contrast image is acquired of a target area. In an embodiment, the first pre-contrast thermal image is a mask image. In an embodiment, the pre-contrast thermal image is an image captured before the spraying of a thermal contrast agent. In an embodiment, the first pre-contrast thermal image serves as a standard for assessing the pathophysiological state of an individual. In an embodiment, the target area is a moist and wet infected area of the body. In a preferred embodiment, the target area is an ulcer. In another embodiment, the target area is a moist and wet area of the internal organs during surgery.

In an embodiment, the target area releases secretions and has a tendency to evaporate water and fluids. In an embodiment, the evaporation of water and fluids keeps the temperature of the target area low as compared to the normal body temperature of an individual. In an embodiment, the first pre-contrast thermal image is acquired when the temperature of the target area is low as compared to the normal body temperature of an individual.

In an embodiment, the first pre-contrast thermal image is acquired by an infrared camera. In an embodiment, the body emits heat in the form of infrared rays. In an embodiment, the infrared rays are detected by the infrared camera and the pre-contrast thermal image is captured. In an embodiment, the thermal emissivity characteristic of the body is used for assessing the pathophysiological state of the individual.

In an embodiment, a video and a time-lapse photograph are recorded. In an embodiment, the pre-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to a surrounding tissue.

At step 304, a transparent barrier is placed over the target area. In a preferred embodiment, the barrier is a transparent film. In an embodiment, the transparent barrier is placed over the wet and moist infected area of the body. In another embodiment, the transparent barrier is placed over the wet and moist area of the internal organs during surgery.

In an embodiment, the barrier is capable of eliminating the evaporation of water from the target area. In an embodiment, the barrier is capable of increasing the temperature of the target area. In an embodiment, the transparent barrier reduces the evaporation of water and fluids from the target area. In an embodiment, the transparent barrier allows the temperature of the target area to be equal to the normal body temperature. In an embodiment, the transparent barrier allows the temperature of the target area to be higher than the normal body temperature in inflammation. In an embodiment, the transparent barrier allows the temperature of the target area to be higher than the normal body temperature in infection. This method is preferred when the target area is an ulcer, since an evaporative fluid such containing alcohol may not be used directly over the target area.

In an embodiment, the transparent barrier is a plastic film, cling film, saran wrap and food wrap. In an embodiment, the transparent barrier is a sheet of plastic and is 12.7 pm thick. In an embodiment, the transparent barrier is made up of materials such as, but not limited to, acrylic, epoxy, epoxy glass fiber, nylon 6, PTFE, PVC and polyethylene. In an embodiment, the transparent barrier has a high thermal conductivity. In an embodiment, the transparent barrier has an emissivity similar to that of the body.

At step 306, a second pre-contrast thermal image is acquired after placing the transparent barrier. In an embodiment, the second pre-contrast thermal image is an image captured after the placement of the transparent barrier. In an embodiment, the barrier is capable of increasing the temperature of the target area. In an embodiment, the transparent barrier reduces the evaporation of water and fluids from the target area. In an embodiment, the transparent barrier allows the temperature of the target area to be equal to the normal body temperature. In an embodiment, the second pre-contrast thermal image is acquired after the temperature recovery of the target area. In an embodiment, a thermal contrast is observed with respect to the first pre-contrast thermal image and the second pre-contrast thermal image. In an embodiment, a thermal contrast is observed between a plurality of points of the target area. In an embodiment, in an ulcer specifically, the plurality of points of the target area includes a base of the target area and an edge of a target area.

In a preferred embodiment, the edge of the target area of the ulcer has an increased temperature as compared to the base of the target area. In an embodiment, the water and the fluids are evaporated from the base of the target area. In an embodiment, the edge of the target area has an increased blood flow as compared to the base of the target area.

In an embodiment, the second pre-contrast thermal image is acquired by the infrared camera. In an embodiment, the body emits heat in the form of infrared rays. In an embodiment, the infrared rays are detected by the infrared camera and the second pre-contrast thermal image is captured.

In an embodiment, the video and the time-lapse photograph are recorded. In an embodiment, the second pre-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to the surrounding tissue.

At step 308, the evaporative fluid is sprayed after placing the barrier. In a preferred embodiment, the evaporative fluid is the thermal contrast agent. In an embodiment, the evaporative fluid is sprayed over the target area. In an embodiment, the evaporative fluid is a mixture of alcohol and water. In a preferred embodiment, the thermal contrast agent contains 65% alcohol and 35% water over the moist and the wet infected area of the individual. In another embodiment, the evaporative fluid is saline. In an embodiment, the cold saline is sprayed onto the moist and wet area during a surgery.

In an embodiment, the evaporative fluid is the water. In an embodiment, the water is heated to a temperature ranging from 40-45° C. In an embodiment, the water is sprayed over the barrier. In an embodiment, the temperature of the target area increases with respect to the normal temperature of the body. In an embodiment, the water establishes the contrast between the target area and the surrounding tissues.

In an embodiment, the volume of the evaporative fluid sprayed onto the target area lies in the range of 15 to 30 ml. In an embodiment, the volume of the evaporative fluid is sufficient enough to cover the size of the target area. For example, for the foot, the evaporative fluid is sprayed sufficient enough to cover the dorsal and plantar surface of the skin. For example, during surgery, the evaporative fluid is sprayed sufficient enough to cover the size of the skin transplant.

In an embodiment, the evaporative fluid is sprayed over the transparent barrier placed on the wet and the moist area of the internal organs during the surgery. In an embodiment, the transparent barrier along with the evaporative fluid is removed from the wet and the moist area of the internal organs. In an embodiment, the temperature recovery of the wet and the moist area of the internal organ is determined. In an embodiment, the spraying of the evaporative fluid establishes a contrast between the wet and the moist area of the internal organs and the surrounding tissue. In an embodiment, the blood flow of the wet and the moist area of the internal organs and the surrounding tissue is determined for establishing the contrast.

In an embodiment, the evaporative fluid is used to enhance the thermal contrast between the areas of higher blood supply and the areas of lower blood supply. In an embodiment, the evaporative fluid enhances the thermal contrast between the base of the target area and the edge of the target area.

At step 310, a post-contrast thermal image is acquired after the evaporation of the evaporative fluid. In an embodiment, the post contrast thermal image is an image captured after the spraying of the thermal contrast agent.

In an embodiment, the evaporation of the evaporative fluid depends on the factors such as, but not limited to, temperature of the room, humidity of the room and evaporation of water. In a preferred embodiment, the post-contrast thermal image is acquired after a pre-determ fined time. In an embodiment, the post-contrast thermal image is acquired after the complete evaporation of the evaporative fluid.

In an embodiment, the post-contrast thermal image is acquired by the infrared camera. In an embodiment, the body emits heat in the form of infrared rays. In an embodiment, the infrared rays are detected by the infrared camera and the post-contrast thermal image is captured.

In an embodiment, the video and the time-lapse photograph are recorded. In an embodiment, the post-contrast thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualization of the target area in relation to the surrounding tissue.

At step 312, the first pre-contrast thermal image, the second pre-contrast thermal image and the post-contrast thermal image are processed. In an embodiment, the target area emits infrared rays. In an embodiment, the target area having a higher blood supply will emit more infrared rays than the area having a lower blood supply. For example, in an ulcer, the edge of the target area requires more supply of blood as compared to the base of the target area having normal blood supply. The edge of the target area will emit more infrared rays as compared to the base of the target area.

At step 314, a thermographic data is obtained from the plurality of points of the target area. In an embodiment, the thermographic data from the plurality of points of the target area enables assessing the pathophysiological state of the individual. In an embodiment, the thermographic data from the plurality of points of the target area enables in assessing the healing of the target area. In an embodiment, the edge of the target area requires more supply of blood as compared to the base of the target area having normal blood supply. The edge of the target area will emit more infrared rays as compared to the base of the target area. In a preferred embodiment, healing of the target area starts from the edge of the target area.

In an embodiment, the emissivity varies for the various tissues of the body. In an embodiment, the emissivity of a tissue varies at different temperatures. In an embodiment, the emissivity of a tissue varies with respect to any pathophysiological change. For example, the emissivity of the bone varies with an injury. The blood flow of the bone varies during the injury. In an embodiment, the change in temperature leads to a change in the emission of heat from the target area.

In an embodiment, the thermal emissivity characteristic of the body is used for assessing the pathophysiological state of the individual. In an embodiment, the individual has a core temperature different than the normal body temperature. In an embodiment, during fever, the core temperature of the individual is higher than the normal body temperature. In an embodiment, the body of the individual absorbs, transmits and reflects infrared radiations. In an embodiment, the contrast is established between the infrared rays emitted from the target area and the infrared rays absorbed, transmitted and reflected by the surrounding tissues.

At step 316, a thermogram is generated. The thermogram is the thermal image generated by the infrared camera. In an embodiment, a composite thermogram is generated. The infrared camera detects the infrared rays, converts the infrared rays into an electrical signal and displays the result as a thermal image. In an embodiment, the thermogram is generated by superimposing the first pre-contrast thermal image, the second pre-contrast thermal image and the post-contrast thermal image. In an embodiment, the superimposed regions are subtracted from the first pre contrast thermal image, the second pre-contrast thermal image and the post-contrast thermal image. In an embodiment, the rate of temperature recovery of the target area is observed. In an embodiment, the thermogram is analysed for assessing the pathophysiological state of the individual. In an embodiment, the pathophysiological state of the individual includes the healing of the target area.

In an embodiment, the thermogram is generated through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the target area in relation to the surrounding tissue.

In an embodiment, the heart rate of the individual is measured for determining the core body temperature of an individual. The heart rate lies in the range from 60-100 beats per minute. In an embodiment, the heart rate increases when the temperature of the individual is increased than the normal body temperature. In an embodiment, 1 degree increase in the body temperature leads to an increase in the heart rate by 10 beats per minute.

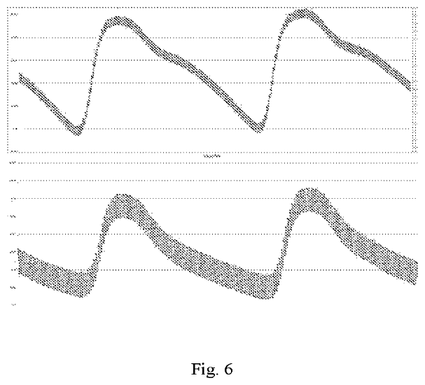

In an embodiment, the heart rate is measured by a device having a camera. In an embodiment, the device includes a mobile phone, a smart watch and a photographic camera. In an embodiment, the heart rate is measured by a non-contact optical technique of photoplethysmography (PPG). In an embodiment, the photoplethysmography (PPG) is used to detect volumetric changes in the blood flow. In an embodiment, the photoplethysmography (PPG) is based on the principle that the blood absorbs more light than the surrounding tissues. In an embodiment, the blood flow affects the reflection of light. In an embodiment, the blood flow is different in systole and diastole. A difference in width of two PPG waves of a subject as shown FIG. 6 indicates change in pathophysiological state.

FIG. 6 shows two pulse sampling done at 1 kHz frequency. The figure shows tracing from the same subject, with one hand at room temperature and the other dipped in cold water and dried. A significant difference may be noted in the thickness of the line in the tracing indicating microvascular changes in the skin due to cold exposure.

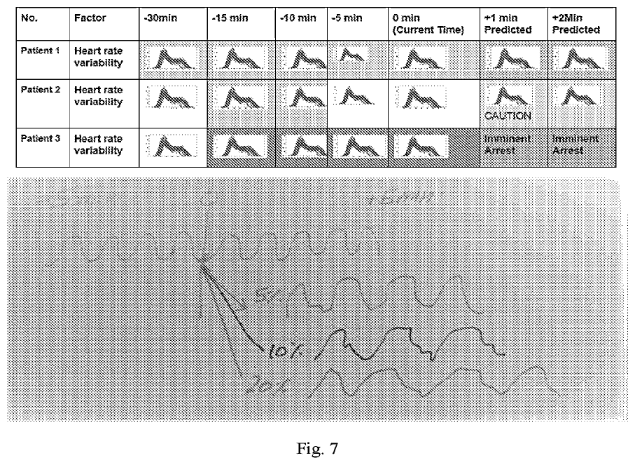

Furthermore, historical pulse data is analyzed using mathematical models and employing an artificial intelligence framework to predict future pulse data, which in turn, are used for what is termed as tracing. The mathematical model is validated by measuring actual subject data following the historical period of time with the predicted data. If there is no change in pathophysiological state of the subject, the difference between the predicted and actual data is minimal. In case there is a significant change in the pathophysiological state of the subject, a divergence in the predicted and actual data is observed. Such a system when employed real time and displays such patterns is indicative of clinical progress (i.e. changes in pathophysiological state of the subject). Such a study or analysis may be carried out for a number of intervals with an alarm or alert going off (or displayed on a display module) as and when a divergence between predicted and actual data, more than a predetermined threshold, is observed. The differences may also be color-coded for attention. For example, a display of red, yellow, and green (as shown in FIG. 7) may respectively be used for danger, need to pay attention, and stable respectively. Other visual and audible means may also be employed to alert a caregiver of the pathophysiological state of the subject.

In an embodiment, the heart rate is measured by placing a finger on the camera of the device. In an embodiment, the flash light of the devices serves as the light source in the visible range for reflection by the blood cells of the individual. In an embodiment, the light reflected is different in systole and diastole.

FIG. 4 illustrates a method (400) of determining the core body temperature of an individual, according to an embodiment. In an embodiment, the heart rate monitoring along with the thermal imaging enables assessing the accurate temperature of the individual. In an embodiment, the heart rate monitoring along with the thermal imaging enables eliminating the possibility of medications taken by an individual for a higher body temperature. The method (400) of determining the core body temperature includes:

At step 402, the heart rate of the individual is measured. The heart rate lies in the range from 60-100 beats per minute. In an embodiment, the heart rate increases when the temperature of the individual is increased than the normal body temperature. In an embodiment, 1 degree increase in the body temperature leads to an increase in the heart rate by 10 beats per minute.

In an embodiment, the heart rate is measured by a device having a camera. In an embodiment, the device includes a mobile phone, a smart watch and a photographic camera. In an embodiment, the heart rate is measured by a non-contact optical technique of photoplethysmography (PPG). In an embodiment, the photoplethysmography (PPG) is used to detect volumetric changes in the blood flow. In an embodiment, the photoplethysmography (PPG) is based on the principle that the blood absorbs more light than the surrounding tissues. In an embodiment, the blood flow affects the reflection of light. In an embodiment, the blood flow is different in systole and diastole. In an embodiment, the blood flow as a function of time is different in different pathophysiological states of a subject.

In an embodiment, the heart rate is measured by placing a finger on the camera of the device. In an embodiment, the flash light of the devices serves as the light source in the visible range for reflection by the blood cells of the individual. In an embodiment, the light reflected is different in systole and diastole.

At step 404, a thermal image is acquired by an infrared camera. In an embodiment, the body emits heat in the form of infrared rays. In an embodiment, the infrared rays are detected by the infrared camera and the thermal image is captured.

In an embodiment, a video and a time-lapse photograph are recorded in real-time. In an embodiment, the thermal image and the video are recorded through a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the thermal images.

At step 406, the core body temperature of the individual is determined. In an embodiment, the core body temperature is determined by analysing the heart rate from step 402 and the thermal images from step 404. In an embodiment, the monitoring of the heart rate enables in assessing the core body temperature of the individual. In an embodiment, the individual has a core temperature different from the normal body temperature. In an embodiment, during fever, the core temperature of the individual is higher than the normal body temperature. In an embodiment, the body of the individual absorbs, transmits and reflects infrared radiations. In an embodiment, the monitoring of the heart rate along with the thermal imaging enables the precise determination of the core body temperature of the individual.

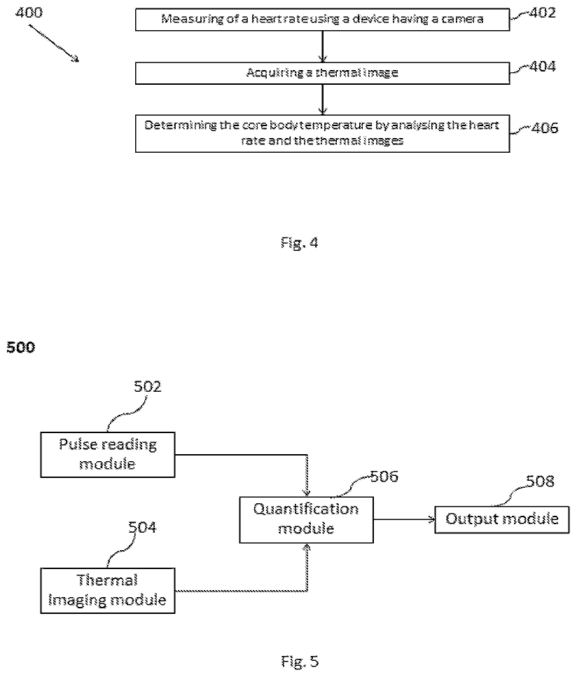

FIG. 5 illustrates a system (500) for determining the core body temperature of an individual, according to an embodiment. In an embodiment, the heart rate monitoring along with the thermal imaging enables assessing the accurate temperature of the individual. In an embodiment, the heart rate monitoring along with the thermal imaging enables eliminating the possibility of medications taken by an individual with a higher body temperature. The system (500) includes a pulse reading module (502), a thermal imaging module (504), a quantification module (506) and an output module (508). The system may be integrated with systems or cameras that rely on visible light images to determine core body temperature to obtain a more precise reading.

The pulse reading module (502) measures the heart rate of the individual. The heart rate lies in the range from 60-100 beats per minute. In an embodiment, the heart rate increases when the temperature of the individual is increased than the normal body temperature. In an embodiment, 1 degree increase in the body temperature leads to an increase in the heart rate by 10 beats per minute.

In an embodiment, the pulse reading module (502) is a device having a camera. In an embodiment, the pulse reading module (502) includes a mobile phone, a smart watch and a photographic camera. In an embodiment, the heart rate is measured by an optical technique of photoplethysmography (PPG). In an embodiment, the photoplethysmography (PPG) is used to detect volumetric changes in the blood flow.

In an embodiment, the photoplethysmography (PPG) is based on the principle that the blood absorbs more light than the surrounding tissues. In an embodiment, the blood flow affects the reflection of light. In an embodiment, the blood flow is different in systole and diastole.

In an embodiment, the heart rate is measured by placing a finger on the camera of the pulse reading module (502). In an embodiment, the flash light of the devices serves as the light source in the visible range for reflection by the blood cells of the individual. In an embodiment, the light reflected is different in systole and diastole. The pulse reading module (502) is capable of assessing the heart beats per minute. In an embodiment, the pulse reading module (502) helps in determining the core body temperature of the individual.

The thermal imaging module (504) is capable of capturing thermal images. In an embodiment, the thermal imaging module (504) is the infrared camera. In an embodiment, the body emits heat in the form of infrared rays. In an embodiment, the infrared rays are detected by the thermal imaging module and the thermal images are captured.

In an embodiment, the thermal imaging module (504) is capable of recording video and the time-lapse photograph in real-time. In an embodiment, the thermal imaging module (504) utilises a multi spectral dynamic imaging (MSX) technique. The multi spectral dynamic imaging (MSX) technique is based on FLIR processors for acquiring the thermal images and the videos in real time. In an embodiment, the multi spectral dynamic imaging (MSX) technique adds visible light to the thermal images. In an embodiment, the visible light allows the better visualisation of the thermal images.

The quantification module (506) communicates with the pulse reading module (502) and the thermal imaging module (504). In an embodiment, the quantification module (506) is capable of processing the thermal images and the heart rate. In an embodiment, the quantification module (506) analyses the input values received from the pulse reading module (502) and the thermal images received from the thermal imaging module (504). In an embodiment, the quantification module (506) of the individual by analysing the input values received from the pulse reading module (502) and the thermal images received from the thermal imaging module (504). In an embodiment, the quantification module (506) analyses the data using computer algorithms of the individual by using the input values received from the pulse reading module (502) and the thermal images received from the thermal imaging module (504). Any disconnect between the predicted pulse for the actual temperature and the thermal image based temperature is flagged for further investigation.

The output module (508) communicates with the quantification module (506) to display the core body temperature of the individual. The output module (508) includes a display for displaying the core body temperature of the individual. The output module (508) may show difference between the expected pulse rate of the subject and the measured pulse rate of the subject.

In an embodiment, a subject is made to pass through a cold ambience and a warm/hot ambience and difference in pulse rate at normal ambience, cold ambience, and warm/hot ambience allows identification of a normal person from an infected (febrile) person with normal temperature achieved by intaking antipyretic medications such as paracetamol.

The present embodiment provides the method for achieving a sharp contrast between the area of higher blood flow and the area of lower blood flow while performing digital subtraction angiography. The present embodiment provides the method for achieving the sharp contrast for different anatomical regions of the body.

The present methods may also be performed by employing a technique of dual spectrum thermal image (DSTA). The emissivity varies for the various tissues of the body and the same tissue varies at different temperatures. The DSTA process includes adjusting the infrared camera at a first wavelength. In an embodiment, the infrared camera detects the infrared rays of the first wavelength and generates a first thermal image. Thereafter, the infrared camera is adjusted at a second wavelength. In an embodiment, the infrared camera will detect the infrared rays of the second wavelength and generate a second thermal image. In an embodiment, the thermogram is generated based on the temperature for the first wavelength and the temperature for the second wavelength. In an embodiment, the first thermal image and the second thermal image are superimposed with each other. In an embodiment, the superimposed regions of the first thermal image and the second thermal image are subtracted. In an embodiment, the technique of dual spectrum thermal image (DSTA) helps in determining the pathophysiological state of an individual.

For example, the first thermal image is captured at a wavelength of 800 nm and the second thermal image is captured at a wavelength of 1200 nm. The first thermal image and the second thermal image is a composite image. In an embodiment, the composite image includes bone, tissue and ring on a finger. The first thermal image and the second thermal image are superimposed with each other. The superimposed regions of the first thermal image and the second thermal image are subtracted. In an embodiment, the regions of the first thermal image and the second thermal image are subtracted to remove the desired tissue.

In an aspect, a computer-implemented method and system is provided to determine the temperature, volume and rate of injecting IV fluid as a contrasting agent, and also to determine a minimum temperature difference between the subject's body and the IV to be injected. The method includes determining the temperature of the individual through a non-contact detection system by comparing the heat radiation emitted during a higher core body temperature with the threshold heat radiation. The method includes determining the temperature of the subject, temperature of the room, humidity at the tissue, humidity in the room, atmospheric pressure sensor and determining the rate of evaporation of water from the target area. The system includes an apparatus including several sensors for measuring temperature and humidity of the room, while simultaneously measuring the temperature of the subject, the humidity of the target area and rate of evaporation at the target area. The system further includes a quantification module, including a microcontroller or a processor, communicating with each of the sensors to compute volume, temperature and rate of injection of IV fluid, which is displayed at a display unit that communicates with the quantification module. For example, the quantification module computes 50 ml of the thermal contrast agent at a temperature of 4° C. based on the parameters assessed by the sensors. The system may further include an actuator that communicates with the quantification module and on the computation of volume, temperature and rate of injection of IV fluid, the actuator allows an opening of the valve [with which the actuator is connected] at the beginning of the tube [near the bottom of the bottle/pouch carrying IV fluid] and automatically starts injecting the IV fluid at the computed rate and varies the volume, rate and temperature, if parameters change drastically. In an embodiment, the bottle/pouch carrying IV fluid is a thermoelectric apparatus having a temperature sensor that communicates with the quantification module to allow modulation of the temperature of the IV fluid.

In another embodiment, the thermal camera can be rotated in a 360° fashion, collecting data and intersection of various thermal patterns and use computerized algorithms to re-create a 3D model of the thermal pattern of the subject. Just like A CT scan, or computed tomography scan is a medical imaging procedure that uses computer-processed combinations of many X-ray measurements taken from different angles to produce cross-sectional (tomographic) images (virtual “slices”) of specific areas of a scanned object, allowing the user to see inside the object without cutting.

In an embodiment, a use of a thermally controlled fluid into a blood vessel of a patient to acquire a post-contrast thermal image of a target area is provided. The use includes acquiring a pre-contrast thermal image of a target area; and injecting a thermally controlled fluid into a blood vessel of the target area, followed by acquisition of a number of post-contrast thermal images after a fixed time interval, and processing the number of thermal images to obtain a thermogram.

In yet another embodiment, a use of an evaporative fluid in obtaining a thermogram, according to methods described herein is provided. The use includes applying the evaporative fluid to a target area after taking a pre-contrast thermal image of the target area, which is then followed by acquisition of post-contrast thermal images after the evaporation of the evaporative fluid from the target area, and processing the number of pre-contrast and post-contrast images to obtain a thermogram. In an embodiment, a use of evaporative fluid in obtaining or performing thermal digital subtraction angiography is provided.

In still another embodiment, a use of thin plastic barrier is provided to perform digital subtraction angiography. In an embodiment, the use of thin plastic barrier is provided to obtain a pre-contrast/post-contrast thermal images to obtain a number of images to obtain a thermogram. This includes first acquiring a first pre-contrast thermal image of the target area; and then using a thin plastic barrier over the target area for acquiring a second pre-contrast thermal image after the temperature recovery of the target area; followed by spraying of an evaporative fluid over the barrier and then acquiring a post-contrast thermal image after the evaporation of the evaporative fluid. The several images are then processed using digital subtraction angiography methods and techniques to visualise target area.

In an embodiment, use of thermal image together with heart rate measurements from a camera is provided to determine core body temperature or pathophysiological state of a person.

The above uses described herein, of evaporative fluid, of film barrier or combination of both to obtain digital subtraction angiography may also be employed in dual spectrum thermal image based methods. As has been explained above, the emissivity varies for the various tissues of the body and the same tissue varies at different temperatures. The DSTA process includes adjusting the infrared camera at a first wavelength. In an embodiment, the infrared camera detects the infrared rays of the first wavelength and generates a first thermal image. Thereafter, the infrared camera is adjusted at a second wavelength. In an embodiment, the infrared camera will detect the infrared rays of the second wavelength and generate a second thermal image. In an embodiment, the thermogram is generated based on the temperature for the first wavelength and the temperature for the second wavelength. In an embodiment, the first thermal image and the second thermal image are superimposed with each other. In an embodiment, the superimposed regions of the first thermal image and the second thermal image are subtracted. In an embodiment, the technique of dual spectrum thermal image (DSTA) helps in determining the pathophysiological state of an individual.

In an embodiment, a contactless method of detecting lies is provided. The lie may be detected from a recorded feed or live feed. In an embodiment, a method of detecting a state of nervousness or stress of an individual or a number of individuals from among a group of a number of individuals is provided. The method includes determining heart rate variability and other pulse parameters using thermal imaging means as described herein together with those provided in the prior art while a person makes a certain statement or provides an answer or answers to certain question or questions. The method of determining pulse, according to an embodiment herein, also includes determining intrapulse variations, which is fluctuation of pulse pressures within a single pulse beat period. This is followed by the method step of determining/computing difference between pulse peaks to determine heart rate variability. These variations/pulse data is compared with the individual's pulse/heart rate variability data of the individual while answering questions that are not a lie or while making statements that are not a lie. The method may further include asking questions or asking the individuals to make statements based on principles of psychological testing for lie detection. While these steps are being performed, a video is recorded, and variability of pulse and heart rate is determined in real time, and the two patterns are compared to determine or predict truthfulness of the statements made.

In an embodiment, a contactless method of detecting alertness levels or emotion levels of an individual or a number of individuals simultaneously is provided. The alertness levels may be detected from a recorded feed or a live feed. The method includes recording or obtaining a video feed of a group of individuals and recording or obtaining pulse wave of each member of the group based on reflection of heat from skin of the individuals, and determining intra-beat variations of each of the pulse data of each of the individuals and followed by determination of inter-beat variations i.e. variations between peaks of multiple pulses and accordingly heart rate variability is determined among other cardiovascular parameters. This data is compared with standardised data of “alert” individuals and a prediction of attentiveness or alertness of an individual from among a group of individuals is then made. In an embodiment, this data may then be used to predict meditative potential of the individual. In an embodiment, this method is applied in a classroom to detect alertness of students.

In an embodiment, a method of detection of suspicious behavior of an individual or more than one individual simultaneously is provided. This method may be applied in public places such as stations or airports. As described above, the heart rate variability data is paired with single intrapulse response and interpulse response for all individuals in a recorded or live feed. Similarly, as described above, this data is compared with the standard data of that of a “non-nervous” individual. This enables prediction of an individual in state of “nervousoness” such as in a patient (during mass medical management as an unmonitored covered settings) or person (for monitoring of terrorism related activities and crowded environments).

In an embodiment, a method of detection of the emotional state of a subject on basis of facial colour by pulse reading and thermal imaging is provided. The method is used to determine emotional state even in absence of facial muscle activation. The method includes capturing images i.e. pre-contrast thermal image of a face of the subject, followed by exposure to the emotional stimulus, briefly, to a change in skin temperature, and capturing a post-contrast thermal image, and preparing a thermogram to visualize spread or patterns of blood flow in veins and arteries on the face i.e. facial colour of the subject, and on the basis of their location or spread across the face, an emotion may be predicted. Emotions are activated by numerous components of the nervous system and manifest differently in different features of the body of a subject. Therefore, it is hypothesized that variations in blood flow or detecting variations in blood flow that may be visible as colour patterns or spread on the surface of skin may enable determining emotions of the subject. For example, the emotion of surprise is going to have colour spread or facial colour change/visualization of it at near eyebrows or temple area or forehead, and the emotion of “happiness” is going to have colour spread or facial colour change/visualization of it at near lips and cheeks. A thermal image pattern as obtained by methods provided herein can provide real time blood flow in the face of the subject and accordingly a prediction of emotion may be made.

The foregoing discussion of the present invention has been presented for purposes of illustration and description. It is not intended to limit the present invention to the form or forms disclosed herein. In the foregoing Detailed Description, for example, various features of the present invention are grouped together in one or more embodiments, configurations, or aspects for the purpose of streamlining the disclosure. The features of the embodiments, configurations, or aspects may be combined in alternative embodiments, configurations, or aspects other than those discussed above. This method of disclosure is not to be interpreted as reflecting an intention the present invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate embodiment of the present invention.

Moreover, though the description of the present invention has included description of one or more embodiments, configurations, or aspects and certain variations and modifications, other variations, combinations, and modifications are within the scope of the present invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A method (100) for performing thermal digital subtraction angiography, the method (100) comprising:
acquiring a pre-contrast thermal image of a target area;
injecting a thermally controlled fluid into a blood vessel at a pre-determined speed;
acquiring a post-contrast thermal image immediately after the injection of the fluid;
acquiring a plurality of post-contrast thermal images after a fixed time interval;
processing of the pre-contrast thermal image, the post-contrast thermal image and the plurality of post-contrast thermal images; and
generating a thermogram.

2. The method (100) of claim 1 further comprises comparing and superimposing the post-contrast thermal image and the plurality of post-contrast thermal images with the pre-contrast thermal image; and subtracting the superimposed portions of the post-contrast thermal image and the plurality of post-contrast thermal images with the pre-contrast thermal image.

3. The method (100) of claim 1, further comprises determining a change in the rate of temperature recovery of the target area; and analysing the thermogram for assessing the pathophysiological state of the subject or individual or assessing the heart rate for determining the core body temperature of the individual.

4. The method (100) of claim 1, wherein the IV fluid is ringer lactate, saline or dextrose solution and the temperature of the IV fluid is different from the normal body temperature.

5. A method (200) for performing thermal digital subtraction angiography, the method (200) comprising:
acquiring a pre-contrast thermal image of a target area;
spraying an evaporative fluid onto the target area;
acquiring a post-contrast thermal image after the evaporation of the evaporative fluid;
acquiring a plurality of post-contrast thermal images after a fixed time interval;
processing of the pre-contrast thermal image, the post-contrast thermal image and the plurality of post-contrast thermal images; and
generating a thermogram.

6. The method (200) of claim 5 further comprises comparing and superimposing the post-contrast thermal image and the plurality of post-contrast thermal images with the pre-contrast thermal image and subtracting the superimposed portions of the post-contrast thermal image and the plurality of post-contrast thermal images with the pre-contrast thermal image.

7. The method (200) as claimed in claim 5, further comprises determining a change in rate of temperature recovery of the target area; and analysing the thermogram for assessing the pathophysiological state of the subject or the person.

8. The method (100) as claimed in claim 5, further comprises assessing the heart rate for determining the core body temperature of the individual.

9. The method (200) as claimed in claim 5, wherein the evaporative fluid is a mixture of alcohol and water.

10. A method (300) of performing digital subtraction angiography, the method (300) comprising:
acquiring a first pre-contrast thermal image of the target area;
placing a thin plastic barrier over the target area;
acquiring a second pre-contrast thermal image after the temperature recovery of the target area;
spraying of an evaporative fluid over the barrier;
acquiring a post-contrast thermal image after the evaporation of the evaporative fluid;
processing of the first pre-contrast thermal image, the second pre-contrast thermal image and the post-contrast thermal image; and
obtaining the thermographic data corresponding to a plurality of points of the target area generating a composite thermogram.

11. The method (300) of claim 10, further comprises comparing and superimposing the first pre-contrast thermal image, the second pre-contrast thermal image and the post-contrast thermal image; and subtracting the superimposed portions of the first pre-contrast thermal image, the second pre-contrast thermal image and the post-contrast thermal image.

12. The method (300) of claim 10, further comprises analyzing the thermogram for assessing the pathophysiological state of the person; and assessing the heart rate for determining the core body temperature of the individual.

13. A method of determining a core body temperature of an individual, the method (400) comprising:
measuring heart rate using a device having a camera;
acquiring a thermal image; and
determining the core body temperature by analysing the heart rate and the thermal images.

14. A method of determining a core body temperature of an individual, the method comprises:
measuring heart rate using a device having a camera;
obtaining a first pre-contrast thermal image;
subjecting the individual to higher and lower temperature than normal core body temperature ambience
obtaining a pulse rate in higher temperature ambience and the lower temperature ambience;
obtaining a post-contrast thermal image each after the individual is subjected to higher and lower temperature ambience;
processing the pre-contrast and post-contrast thermal images by digital subtraction;
determining core body temperature from thermal images and heart rate variability.

15. A system for determining a core body temperature of an individual, the system comprises:
a pulse reading module capable of determining the heart rate;
a thermal imaging module capable of capturing thermal images of a target area;
a quantification module communicates with the pulse reading module and the thermal imaging module, wherein the quantification module is capable of processing the heart rate and the thermal images for determining the core body temperature; and
an output module communicates with the quantification module, wherein the output module is capable of displaying the core body temperature.

16. A method of performing dual spectrum thermal image (DSTA) analysis on a subject to determine pathophysiological state of an individual comprises:
obtaining a first thermal image at a first wavelength in a higher temperature ambience;
obtaining a second thermal image at a second wavelength in a lower temperature ambience;
generating a thermogram based on the temperature for the first wavelength and the temperature for the second wavelength;
superimposing the first thermal image and the second thermal image and subtracting the superimposed regions.

17. A contactless method for detecting lies of an individual, the method comprising
determining heart rate variability and pulse parameters using thermal and visual images;
determining pulse to detect intra-pulse variations within a single pulse beat period;
determining/computing difference between pulse peaks to determine heart rate variability;
comparing intra-pulse variations with the individual's pulse/heart rate variability data while answering questions that are not a lie or while making statements that are not a lie; and
comparing patterns to make a prediction of lie.

18. A contactless method for detecting alertness of an individual, the method comprising:
determining heart rate variability and pulse parameters using thermal and visual images;
determining pulse to detect intra-pulse variations within a single pulse beat period;
determining/computing difference between pulse peaks to determine heart rate variability.
comparing intra-pulse variations with the individual's pulse/heart rate variability data of an alert individual; and
comparing patterns to make a prediction of alertness.

19. A contactless method for detecting an individual in state of nervousness, the method comprising:
determining heart rate variability and pulse parameters using thermal and visual images;
determining pulse to detect intra-pulse variations within a single pulse beat period;
determining/computing difference between pulse peaks to determine heart rate variability.
comparing intra-pulse variations with the individual's pulse/heart rate variability data of a non-nervous individual; and
comparing patterns to make a prediction of nervousness.

20. A method of detection of the emotional state of an individual on the basis of facial blood flow, comprising:
Obtaining pre-contrast thermal image of a face of the subject;
exposure to the emotional stimulus
capturing a post-exposure thermal image;
preparing a thermogram to visualize spread or patterns of blood flow in veins and arteries on the face; and
and on the basis of their location or spread of blood flow patterns across the face, an emotion is predicted.

21. The method as claimed in 18, 19 or 20 wherein the images are still images, or recorded video or live video.
